(19) United States
(12) Patent Application Publication
(10) Pub. No.: US 2026/0325057 A1
(43) Pub. Date: Oct. 22, 2026

(54) MATERNAL HEALTH MONITORING PATCH

(71) Applicant: Adanna Johnson, Catonsville, MD (US)

(72) Inventor: Adanna Johnson, Catonsville, MD (US)

(21) Appl. No.: 19/535,840

(22) Filed: Feb. 10, 2026

Related U.S. Application Data
(60) Provisional application No. 63/757,261, filed on Feb. 11, 2025.

(51) Int. Cl. A61B 5/00; A61B 5/01; A61B 5/02; A61B 5/0205; A61B 5/021; A61B 5/024; A61B 5/1455

(52) U.S. Cl. CPC A61B 5/4362; A61B 5/01; A61B 5/02042; A61B 5/02055; A61B 5/021; A61B 5/02444; A61B 5/14552; A61B 5/6823; A61B 5/6824; A61B 5/6826; A61B 5/6833; A61B 5/7235; A61B 5/742; A61B 5/7475; A61B 2562/164

(57) ABSTRACT

A health tracking system comprising a heart rate monitor, a fetal doppler, a blood pressure monitor, an oximeter, an onboard processor, and an external AI wherein the onboard processor processes sensor data and performs feature extraction, and the external AI is configured to perform at least one of pattern recognition, sequence based temporal analysis, higher level temporal analysis and a combination of these is disclosed. A method of monitoring the health of an organism comprising providing a health monitoring system comprising a heart rate monitor, a fetal doppler, a blood pressure monitor, an oximeter, an onboard processor, and an external AI wherein the onboard process is configured to process sensor data and perform feature extraction, and the external AI is configured to perform pattern recognition, sequence based temporal analysis, and higher level temporal analysis, and using the health tracking system to track the health of the organism is disclosed.

This application claims priority to U.S. Provisional Application No. 63/757,261 filed on Feb. 11, 2025, the entirety of which is incorporated herein by reference.

### FIELD

This invention relates generally to medical sensors. More particularly, the present invention relates to pregnancy monitoring medical sensors.

### BACKGROUND

Maternal health complications, including pre-eclampsia, gestational hypertension, postpartum hemorrhage and lactation difficulties require continuous monitoring to reduce risks and improve outcomes. The rate of pre-eclampsia in the US has been steadily increasing and now effects 1 in 12 pregnancies (Biologyinsights.com) while 60 to 95 of every 1,000 women giving live birth experience gestational hypertension (childstats.gov) and 19.7% of women experience postpartum hemorrhage (contemporaryobgyn.net).

Traditionally, these issues are monitored for and dealt with through expensive periodic clinical visits that may or may not detect early warning signs. Additionally, new and expectant mothers often experience gaps in care, and limited medical assistance outside of clinical visits.

Thus, there is a need for a smart, non-invasive, real-time monitoring system that offers continuous, intelligent health tracking that gives birth mothers and health practitioners critical insights into the birth mother's and the baby's health and well-being.

### SUMMARY

A health tracking system comprising a heart rate monitor, a fetal doppler, a blood pressure monitor, and an oximeter is disclosed. The heart rate monitor may comprise at least one 4 cm×4 cm biometric patch. The blood pressure monitor may comprise at least one 2.5 cm×2.5 cm biometric patch. The oximeter may comprise at least 1 cm×1 cm biometric patch. The fetal doppler may comprise at least one 4 cm×4 cm biometric patch. The health tracking system may further comprise temperature sensors. The temperature sensors may comprise at least one biometric patch. The health tracking system may further comprise a processor and an input/output system configured to provide real time data visualization and predictive analysis. The health tracking system may be configured to track contraction frequency and duration as well as monitor labor progression. The health tracking system may be configured to detect at least one of hemorrhage, infection, lactation challenges or a combination of these. The health tracking system may be configured to track milk production and provide personalized recommendations for lactation support.

A health tracking system comprising a heart rate monitor, a fetal doppler, a blood pressure monitor, an oximeter, an onboard processor, and an external AI wherein the onboard processor is configured to process sensor data and perform feature extraction, and the external AI is configured to perform at least one of pattern recognition, sequence based temporal analysis, higher level temporal analysis and a combination of these is also disclosed. The external AI may comprise at least one of a conventional neural network, a long short-term memory network and a combination of these. The conventional neural network may be configured to perform pattern recognition and the long short-term memory network may be configured to perform at least one of sequence based temporal analysis, higher level temporal analysis and a combination of these.

A method of monitoring the health of an organism comprise providing a health monitoring system comprising a heart rate monitor, a fetal doppler, a blood pressure monitor, an oximeter, an onboard processor, and an external AI wherein the onboard process is configured to process sensor data and perform feature extraction, and the external AI is configured to perform pattern recognition, sequence based temporal analysis, and higher level temporal analysis, and using the health tracking system to track the health of the organism is also disclosed.

### Notes on Construction

The use of the terms “a”, “an”, “the” and similar terms in the context of describing the invention are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. The terms “substantially”, “generally” and other words of degree are relative modifiers intended to indicate permissible variation from the characteristic so modified. The use of such terms in describing a physical or functional characteristic of the invention is not intended to limit such characteristic to the absolute value which the term modifies, but rather to provide an approximation of the value of such physical or functional characteristics.

Terms concerning attachments, coupling and the like, such as “connected” and “interconnected”, refer to a relationship wherein structures are secured or attached to one another either directly or indirectly through intervening structures, as well as both moveable and rigid attachments or relationships, unless specified herein or clearly indicated by context. The term “operatively connected” is such an attachment, coupling or connection that allows the pertinent structures to operate as intended by virtue of that relationship.

The use of any and all examples or exemplary language (e.g., “such as” and “preferably”) herein is intended merely to better illuminate the invention and the preferred embodiment thereof, and not to place a limitation on the scope of the invention. Nothing in the specification should be construed as indicating any element as essential to the practice of the invention unless so stated with specificity.

### BRIEF DESCRIPTION OF THE DRAWINGS

Further advantages of the invention are apparent by reference to the detailed description when considered in conjunction with the figures, which are not to scale to more clearly show the details, wherein like reference numerals represent like elements throughout the several views, and wherein:

FIG. 1 shows the sensors associated with a maternal health tracking system, according to an embodiment of the present invention;

FIG. 2 shows a general outline of data analysis according to an embodiment of the present invention;

FIG. 3 shows an alternative outline of data analysis according to an embodiment of the present invention;

FIG. 4 shows an alternative outline of data analysis according to an embodiment of the present invention;

FIG. 5 shows an alternative outline of data analysis according to an embodiment of the present invention;

FIG. 6 shows an alternative outline of data analysis according to an embodiment of the present invention; and

FIG. 7 shows a possible graphical user interface that could be associated with an app tied to the maternal health tracking system, according to an embodiment of the present invention.

### DETAILED DESCRIPTION

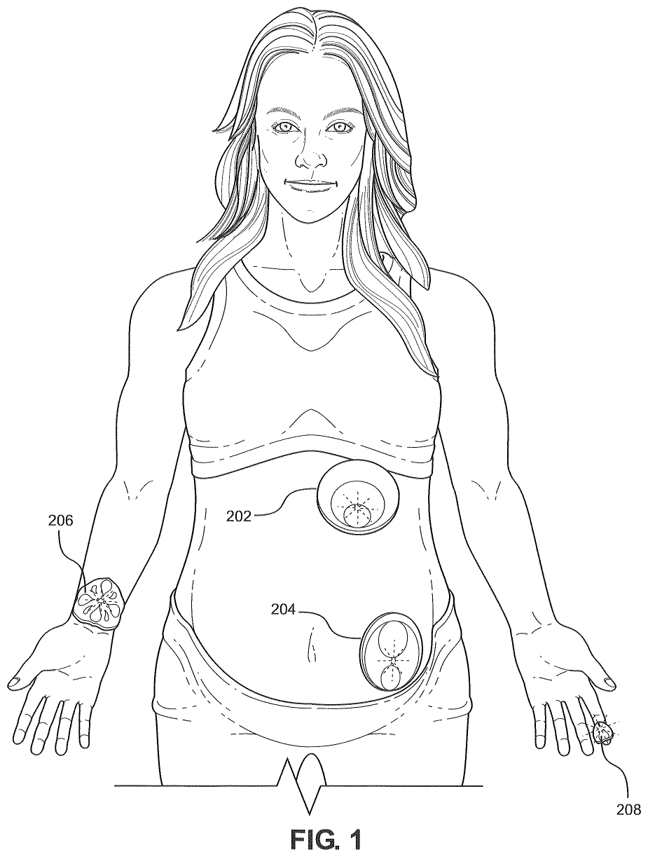

A maternal health tracking system 200 shown in FIG. 1 comprising a heart rate monitor 202, a fetal doppler 204, a blood pressure monitor 206, and an oximeter 208, is disclosed. The heart rate monitor 202 may comprise at least one 4 cm×4 cm biometric patch. The oximeter 208 may comprise at least one 1 cm×1 cm biometric patch. The blood pressure monitor may comprise at least one 2.5 cm×2.5 cm biometric patch. The fetal doppler 204 may comprise at least one 4 cm×4 cm biometric patch. The health tracking system 200 may additionally include temperature sensors (not shown). The temperature sensors may comprise at least one biometric patch. The biometric patches included in the maternal health tracking system 200 may further comprise medical grade silicone, graphene-based conductive sensors and stretchable polymer substrates.

The health tracking system 200 may further comprise a processor and an input/output system configured to provide real time data visualization and predictive analytics. The health tracking system 200 may be configured to tract contraction frequency and duration as well as monitor labor progression. The health tracking system 200 may be configured to detect, hemorrhage, infection, lactation challenges or a combination of these conditions. The health tracking system 200 may be further configured to track milk production and provide personalized recommendations for lactation support.

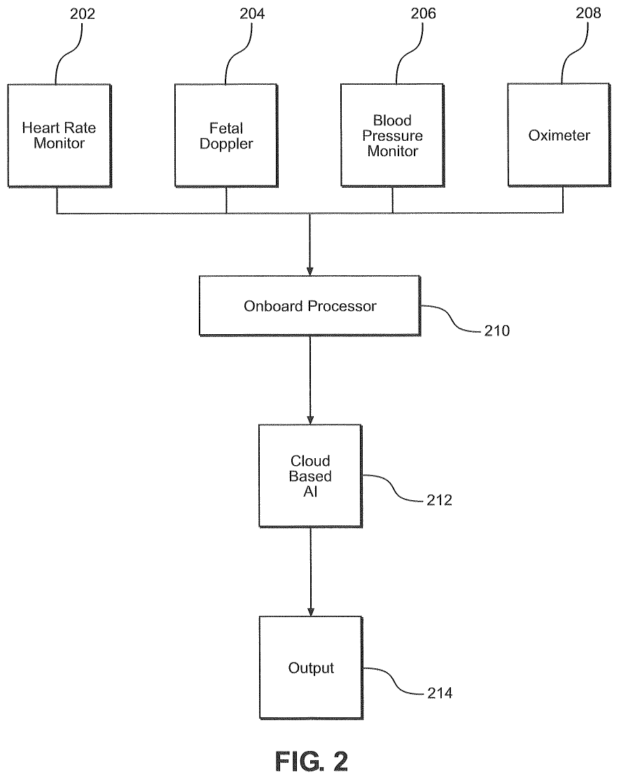

The patches in health tracking system 200 may send, as shown in in FIG. 2, various data to onboard processor 210. This data may be sent through wires, wirelessly, or by any other means known in the art of signal transmission. The patches in health tracking system 200 may send ECG, PPG, fetal heartbeat and spectral reflections, uterine muscle activity, skin temperature, skin conductance/impedance, accelerometer or gyroscope and device placement information to onboard processor 210. The heart rate monitor 202 may send an ECG signal. The fetal doppler 204 may transmit a fetal heartbeat and spectral reflections. The oximeter patch 208 may transmit PPG signals. The ECG signal may be a multi-lead or single-lead waveform at 250-1000 Hz. The PPG signal may use green, red, or infrared channels at 25-200 Hz and provide HRV and SpO₂ estimates. The fetal heartbeat audio and spectral reflections may be sampled at 4-48 kHz. The fetal heartbeat audio and spectral reflections may be processed by the processor into power spectral densities. The uterine muscle activity may be low-frequency contraction signals. The uterine muscle activity may be sampled at 200-1000 Hz. The skin temperature may have 0.01-0.1 °C resolution and may be sampled at 0.1-1 Hz. The skin conductance and impedance may be sampled at 1-10 Hz and used as proxies for lactation conductivity and hydration.

Any combination of the heart rate monitor 202, fetal doppler 204, blood pressure monitor 206, oximeter 208, and any temperature pressure sensors present may perform analog pre-filtering, anti-aliasing, ADC conversion, and event buffering. Any combination of the heart rate monitor 202, fetal doppler 204, blood pressure monitor 206, oximeter 208, and any temperature pressure sensors present may perform adaptive noise removal using, for example but not limited to, bandpass, or notch filters. If an accelerometer is present the accelerometer may detect motion artifacts by removing or marking segments. Any combination of the heart rate monitor 202, fetal doppler 204, blood pressure monitor 206, oximeter 208, and any temperature pressure sensors present may perform basic feature extraction including but not limited to R and/or PPG peak beat detection, instantaneous HR and RR intervals, SpO₂ estimation, temperature delta measurement, and EMB burst detection.

Onboard processor 210 may send information that is then analyzed by cloud based AI 212 to produce output 214.

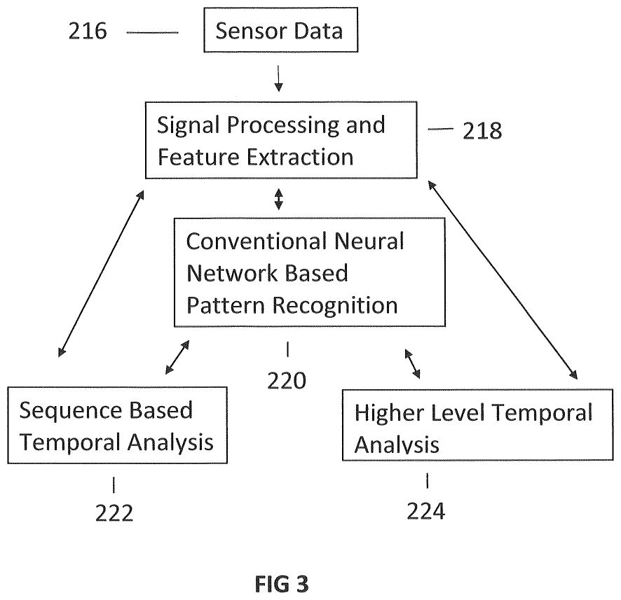

Health tracking system 200 may operate, as outlined in FIG. 3, with heart rate monitor 202, fetal doppler 204, blood pressure monitor 206, oximeter 208, and any temperature and pressure sensors present gathering sensor data 216 and onboard processor 210 performing signal processing and feature extraction 218. Cloud based AI 212 may perform conventional neural network based pattern recognition 220, sequence based temporal analysis 222, and/or higher level temporal analysis 224.

The onboard processor may reject secondary artifacts and score channels. The onboard processor may additionally perform lightweight anomaly detection using threshold flags for immediate user feedback and use a local decision engine to escalate urgent events. The onboard processor may also perform multichannel synchronization by, for example but not limited to, aligning ECG, PPG, Doppler, and/or EMG data by timestamp.

A cloud based AI may combine sensor readings in a hierarchical sensor-fusion pipeline using multimodal architectures that ingest synchronized feature tensors and output event probabilities along with interpretable feature attributions. Signals may be processed in ordered layers of increasing abstraction, confidence and clinical meaning where higher-level inferences depend on the successful validation of lower level insights.

Physiological signals may be processed in successive layers of feature extraction, domain-specific fusion, and cross domain correlation. Higher level event probabilities may be generated only after validation and concordance of lower-level features.

A cloud based AI may combine HR, HRV time and frequency, pulse amplitude and waveform morphology metrics from the heart rate monitor 202. An AI in the cloud may combine power spectral peaks of the fetal heart rate, harmonics, and SNR metrics from the fetal doppler 204. An AI in the cloud may further combine the EMG envelope, RMS spectral energy, burst frequency, and the contraction onset slope. The processor may also combine the baseline, delta, and rate of change of the temperature and conductance data.

The processor may combine the pulse transit time from the ECG and the PPG to provide a cuffless blood pressure estimate. The PPG morphology and the HRV may be combined to give an autonomic state, and the EMG and uterine pressure proxies, such as but not limited to, EMG envelope amplitude, EMG burst duration, EMG burst frequency, EMG activation slope, Integrated EMB signal energy, trends in the inter-contraction interval, contraction regularity, temporal alignment of EMG bursts with maternal cardiovascular response, correlation of EMG activity with accelerometer-derived abdominal micro-motion, and progressive changes in EMG characteristics across successive contractions may be combined to give contraction intensity metrics.

The cloud based AI may make predictions and analyze data using a mixture of classical DSP, statistical models, and modern machine learning. The processor may rely on deterministic and DSP rule based layers for robust heartbeat detection and elementary fillers when machine learning confidence is low. Convolutional Neural Networks may be used for waveform pattern recognition of ECG morphology and Doppler spectrograms. Contraction timing and fetal heart rate trends may be analyzed and predicted using recurrent and transformer temporal models model sequences. Gradient-boosted trees and random forests may be used for tabular risk scoring combining sensor readings and demographics. Abrupt physiological shifts such as maternal hypotension episodes and preeclampsia trends may be detected using Bayesian changepoint detection and hidden Markov models. Autoencoders and isolation forests may be used to surface novel rare patterns. Ensemble meta-classifiers may combine outputs into unified alerts including probabilities and confidences.

The system may analyze changes in skin conductance, tissue impedance and temperature measured at breast-adjacent locations. The system may combine this information with cardiovascular and hydration indicators to estimate lactation activity. The system may record estimations of lactation activity over time. The system may predict breast engorgement or milk stasis risk. The system may measure occurrence of milk let-down events. The system may measure hydration-limited lactation patterns. The system may estimate increased likelihood of mastitis or localized inflammation.

The system may indicate if lactation is actively occurring, temporarily reduced or delayed, as well as initiating early, by detecting sustained decreases in tissue impedance, increased skin conductance, and mild localized temperature elevation. The system may combine this information with, measured by the system or otherwise, postpartum cardiovascular changes to improve accuracy.

Lactation activity over time may be measured using trend analysis of conductance and impedance data over periods of time. This data may be normalized to the individuals baseline and hydration state.

Breast engorgement and milk statis risk may be predicted by detecting a rapid impedance decrease corelated with a rising temperature and conductance, measured by the system or otherwise, that do not resolve after feeding or pumping cycles.

Let down associated physiological responses may be estimated by detection of short-term conductance spikes and temperature shifts that are temporally correlated with maternal autonomic changes including but not limited to HRV shifts associated with oxytocin release.

Hydration limited lactation patterns may be estimated by determining if reduced lactation activity correlates with dehydration, measured by the system or otherwise and if hydration improvements, measured by the system or otherwise, precede increased lactation indicators including but not limited to those previously discussed.

The accelerometer and gyroscope may measure motion context, posture, and activity level. The accelerometer and gyroscope may measure this activity at 50-200 Hz. The processor may combine any combination of the above with one or more metadata and context data such as but not limited to gestational age, maternal history, medications, prior labs, device placement, timestamps, and any user-reported events such as but not limited to contraction start/stop symptoms.

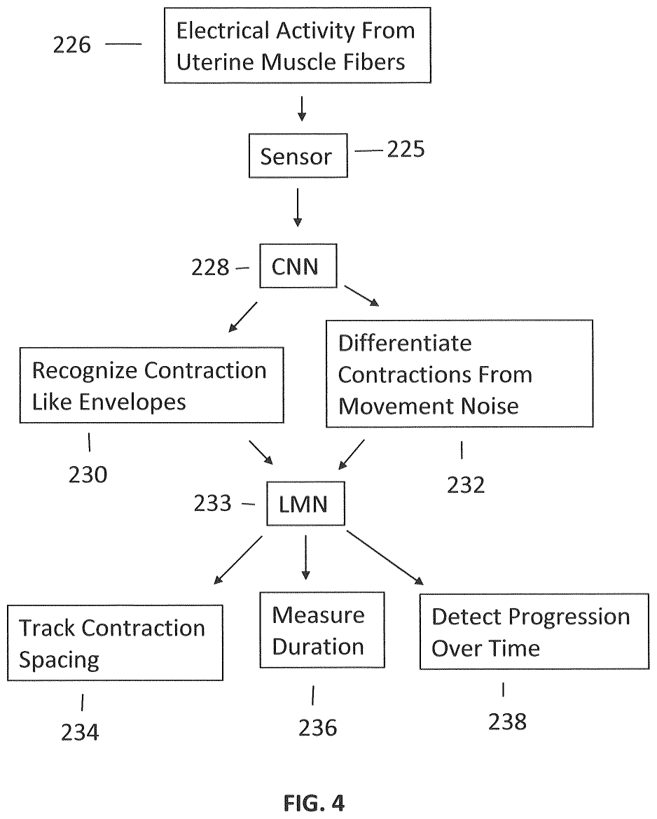

As outlined in FIG. 4, health monitoring system 200, using sensors 225, may measure electrical activity generated by uterine muscle fibers 226. A convolutional neural network (“CNN”) 228 may recognize contraction-like envelopes 230 and differentiate contractions from movement noise 232. A long short-term memory network 233 may track contraction spacing 234, measure duration 236, and detect progression over time 238.

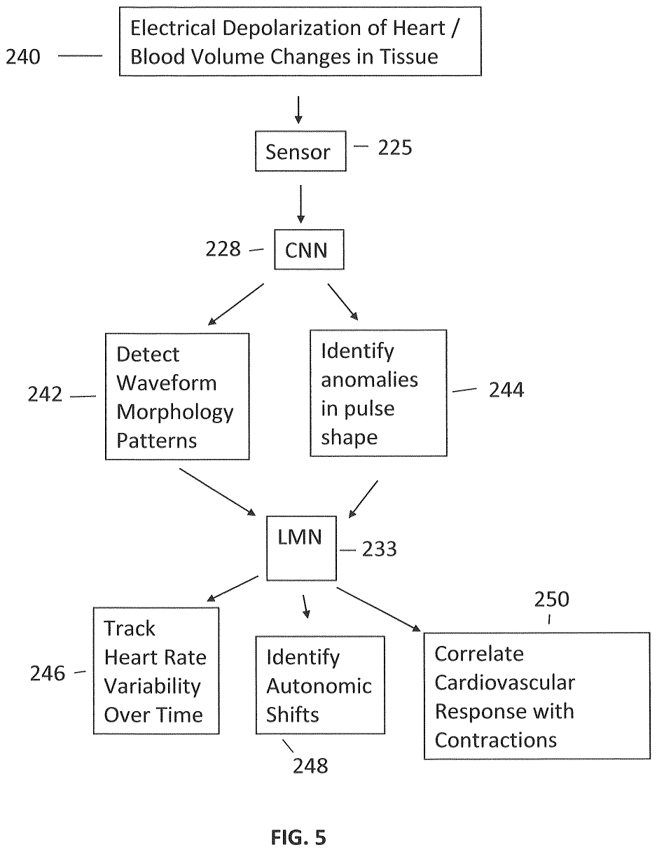

As outlined in FIG. 5, health monitoring system 200, using sensors 225, may measure electrical depolarization of the heart and/or blood volume changes in tissues 240. Conventional neural network 228 may detect waveform morphology patterns 242 and identify anomalies in pulse shape 244. Long short-term memory network 233 may track heart rate variability over time 246, identify autonomic shifts 248, and correlate cardiovascular response with contractions 250.

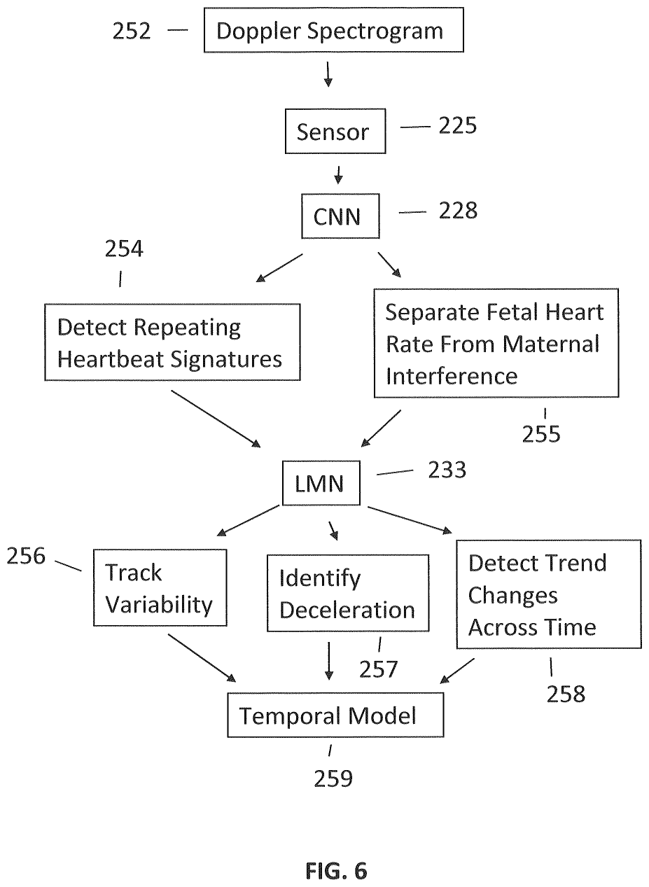

As outlined in FIG. 6, health monitoring system 200, using sensors 225, may measure doppler spectrogram 252. Conventional neural network 228 may detect repeating heartbeat signatures 254 and separate the fetal heart rate from maternal interference 255. Long short-term memory network 233 may track variability 256, identify deceleration 257, and detect trend changes across time 258. Cloud based AI 212 may use this information to create temporal model 259.

The AI may be trained on data with binary and multi class labels such as but not limited to contraction start, stop, and intensity; fetal hear rate category (normal, bradycardia, tachycardia, variable decelerations); maternal hypertensive event, hypoxia episodes; lactation anomalies such as engorgement; suspected infection; gestational diabetes risk markers, along with temporal labels.

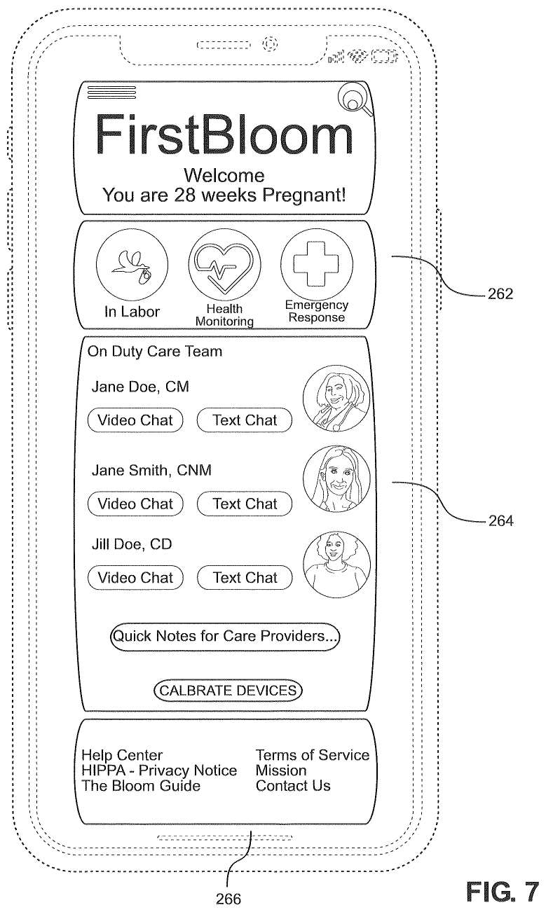

Health monitoring system 200 may further comprise an app with a graphical user interface 260 as shown in FIG. 7. Graphical user interface 260 may comprise menu items 262 that allow a user to indicate if the user is in labor, or needs an emergency response, along with providing access to health monitoring data. The users care team 264 may be collated with text and voice contact capabilities. The graphical user interface may also include links to a help center, terms of service, a HIPPA privacy notice, and a guide to pregnancy 266.

In use the health tracking system 200 may create a preliminary alert that is visualized to the user. A cloud based AI may process synchronized multi-session data and model ensembles to produce a validated alert. Validated alerts are given a severity score and routed to either the in-app device, a remote triage nurse or doula, or an immediate call to an on-call physician and/or emergency services dispatch.

False alarms may be managed by repeated confirmation logic, multi-sensor corroboration, and clinician feedback loops. Users may also be able to provide a false alarm notification that is fed back into the model for model improvement.

In use, the heart rate monitor 202 is generally placed in the thoracic region or the upper abdomen and detects the maternal heartrate, heart rate variability, arrhythmia, baseline cardiovascular stress, uterine muscle activity, and low-frequency electrical signals associated with myometrial activation. The fetal doppler 204 is typically placed on the lower abdomen and aligned with fetal position. Fetal doppler 204 typically transmits a fetal heartbeat signal that is acquired via doppler ultrasound or acoustic sensing, along with spectral reflections, and/or doppler frequency shifts and spectral power data. Oximeter 208 is generally placed on a finger, a wrist, or a well-perfused peripheral site and transmits a multi-wavelength ppg signal.

### EXAMPLES

The following are provided as examples only and should not be used to limit the coverage of the disclosure which is contained in the claims and the detailed description.

### Contraction Detection

The EMG envelope can be combined with signal energy features, and a machine learning model can identify contraction onset, duration and estimated intensity. Motion artifacts are discounted using maternal heart rate and movement data, and contraction timing metrics are produced.

### Fetal Distress

Fetal heart rate beats and trendlines are detected using convolutional neural network processed doppler spectrograms. Fetal doppler 204 may measure frequency shifts caused by moving fetal cardiac tissue. A convolutional neural network may detect repeating heartbeat signatures, separate fetal heart rate from maternal interference. A long short-term memory network may track variability, identify deceleration, and detect trend changes across time. Temporal models detect late, variable or prolonged decelerations associated with maternal hypotension, hypoxia, or uterine hyperstimulation. Alert severity is derived from decel depth, duration, and persistence.

### Preeclampsia Signature

Preeclampsia may be detected using a multivariate trend model. A progressive elevation in systolic/diastolic estimates from the PTT and BP calibration, abnormal HRV changes, sudden changes in user entered weight/edema proxies may be combined with urine protein metadata. Clinician review is prompted when Bayesian changepoint detection signals an abnormal rise.

### Postpartum Hemorrhage Early Warning

An urgent clinician cascade may be triggered by rapid heart rate increases, falling PTT derived blood pressure estimates, cooling skin temperature, and uterine contraction abnormalities.

### Example in Use

To identify preeclampsia, the health tracking system 200 may detect uteroplacental insufficiency with evolving fetal distress by recording EMG contraction bursts and doppler decelerations during home labor. The on patch digital signal processing detects a contraction envelope and transmits timestamped data. The cloud ensemble confirms repeated late decelerations and correlates with an SpO₂ drop and a maternal bloop pressure trending upward. The system then generates a high-priority alert with waveform snippets and calls the remote triage nurse.

What is claimed is:

1. A health tracking system comprising:
a heart rate monitor,
a fetal doppler,
a blood pressure monitor, and
an oximeter.

2. The health tracking system of claim 1 wherein the heart rate monitor comprises at least one 4 cm×4 cm biometric patch.

3. The health tracking system of claim 1 wherein the oximeter comprises at least one 1 cm ×1 cm biometric patch.

4. The health tracking system of claim 1 wherein the blood pressure monitor comprises at least one 2.5 cm×2.5 cm biometric patch.

5. The health tracking system of claim 1 wherein the fetal doppler comprises at least one 4 cm×4 cm biometric patch.

6. The health tracking system of claim 1 wherein the health tracking system is configured to track contraction frequency and duration as well as monitor labor progression.

7. The health tracking system of claim 1 wherein the health tracking system is configured to detect at least one of hemorrhage, infection, lactation challenges or a combination of these.

8. The health tracking system of claim 1 wherein the health tracking system is configured to track milk production and provide personalized recommendations for lactation support.

9. The health tracking system of claim 1 further comprising temperature sensors.

10. The health tracking system of claim 9 wherein the temperature sensors comprise at least one biometric patch.

11. The health tracking system of claim 1 further comprising a processor and an input/output system configured to provide real time data visualization, and predictive analytics.

12. The health tracking system of claim 1 wherein the heart rate monitor comprises at least one 4 cm×4 cm biometric patch, the fetal doppler comprises at least one 4 cm×4 cm biometric patch, the blood pressure monitor comprises at least one 2.5 cm×2.5 cm biometric patch, and the oximeter comprises at least one 1 cm×1 cm biometric patch.

13. The health tracking system of claim 12 wherein the biometric patches further comprise medical grade silicone, graphene-based conductive sensors and stretchable polymer substrates.

14. The health tracking system of claim 1 further comprising a pregnancy stage tracker that offers tailored medical information and weekly wellness tips.

15. A health tracking system comprising:
a heart rate monitor,
a fetal doppler,
a blood pressure monitor,
an oximeter,
an onboard processor, and
an external AI;
wherein the onboard processor is configured to process sensor data and perform feature extraction, and the external AI is configured to perform at least one of pattern recognition, sequence based temporal analysis, higher level temporal analysis and a combination of these.

16. The health tracking system of claim 15 wherein the external AI comprises at least one of a conventional neural network, a long short-term memory network and a combination of these.

17. The health tracking system of claim 16 wherein the conventional neural network is configured to perform pattern recognition.

18. The health tracking system of claim 16 wherein the long short-term memory network is configured to perform at least one of sequence based temporal analysis, higher level temporal analysis and a combination of these.

19. The health tracking system of claim 15 wherein the external AI is configured to track uterine contraction spacing associated with pregnancy, measure uterine contraction duration associated with pregnancy, and detect uterine contraction progression associated with pregnancy over time.

20. A method of tracking the health of an organism comprising:
providing a health tracking system comprising:
a heart rate monitor,
a fetal doppler,
a blood pressure monitor,
an oximeter,
an onboard processor, and
an external AI;
wherein the onboard processor is configured to process sensor data and perform feature extraction, and the external AI is configured to perform pattern recognition, sequence based temporal analysis, and higher level temporal analysis;
using the health tracking system to track the health of the organism.
